(19) United States
(12) Patent Application Publication
(10) Pub. No.: US 2012/0087464 A1
(43) Pub. Date: Apr. 12, 2012

(54) MULTI-SOURCE LOW DOSE X-RAY CT IMAGING APARATUS

(75) Inventors: William K. McCroskey, Solon, OH (US); Timothy W. Milliff, Montville, OH (US); Christ H. Heipp, Montup, OH (US)

(73) Assignee: FMI TECHNOLOGIES, INC., Akron, OH (US)

(21) Appl. No.: 13/269,602

(22) Filed: Oct. 9, 2011

Related U.S. Application Data
(60) Provisional application No. 61/391,621, filed on Oct. 9, 2010.

(51) Int. Cl. A61B 6/03

(52) U.S. Cl. 378/9

(57) ABSTRACT

Some embodiments include a low-dose CT apparatus. Such an apparatus can comprise a plurality of x-ray sources disposed on a gantry and spaced apart along a z-axis. The sources may be configured to produce substantially overlapping fan beams, wherein overlap is substantially complete at a detector surface. Some embodiments also include a controller in electronic communication with the plurality of x-ray sources. The controller may be adapted to switch the plurality of x-ray sources between on and off states such that only one x-ray source is in an on state at any time. Furthermore, the controller circuit may be in electronic communication with at least one x-ray detector and may be adapted to synchronize the x-ray detector with the plurality of x-ray sources such that detected x-rays can be matched to an x-ray source.

### I. CROSS-REFERENCE TO RELATED APPLICATIONS

This application claims priority to U.S. Provisional Patent Application No. 61/391,621 filed Oct. 9, 2010 and now pending, and which is incorporated by reference in its entirety.

### II. BACKGROUND OF THE INVENTION

A. Field of Invention

This invention generally relates to low-dose x-ray sources for x-ray computed tomography systems.

B. Description of the Related Art

In general, x-ray computed tomography (CT) is conducted using an x-ray source, such as a rotating anode source, mounted on a gantry with one or more x-ray scintillation detectors disposed in an opposing orientation. The gantry typically includes a central aperture for receiving a patient such that the patient is disposed between the source and detector(s). Thus, an x-ray beam passes from the source through the patient and an attenuated x-ray intensity pattern is measured by the detector. Typically, the gantry rotates the source and detectors about the patient. Data collected in this manner can be used to reconstruct a two-dimensional slice image of the patient using known image reconstruction algorithms such as filtered back projection. Similarly, a three-dimensional image can be created by simultaneously rotating and translating the source relative to the patient in a helical pattern.

Some tissues attenuate x-rays more than others, so images can be formed from transmitted x-rays showing contrasting structures within the body. For instance, bone attenuates x-rays more efficiently than soft tissue; therefore, bones are readily imaged. Other tissues and organs, such as blood vessels, are more difficult to image due to their similarity in attenuation properties relative to the surrounding tissue. In such cases, contrast agents can be administered to the patient to make target organs and/or tissues more opaque to x-rays. Some such contrast agents include barium or iodine compounds.

During data collection it is generally necessary for the subject to remain still. Movements of the portion of the body being imaged can cause blurring and degradation of image quality. Thus, it is particularly challenging to image the heart, which moves significantly during contraction, i.e. systole, and also due to the subject's breathing. It is known to use EKG gating methods to correct for such movement.

The particular beam intensity necessary for a given imaging procedure depends on the structures being imaged. Examples of typical radiation doses experienced by patients are shown according to procedure in Table 1.

TABLE 1

| Examination | Dose (mSv) | Equivalent Chest X-Rays |
|---|---|---|
| Extremities (e.g. knee, ankle) | 0.01 | 0.5 |
| Chest | 0.02 | 1 |
| Skull | 0.1 | 5 |
| Cervical Spine | 0.1 | 5 |
| Dorsal Spine | 1.0 | 50 |
| Lumbar Spine | 2.4 | 120 |
| Hip | 0.3 | 15 |
| Pelvis | 1.0 | 50 |
| Abdomen | 1.5 | 75 |
| Esophagus | 2.0 | 100 |
| Stomach/Duodenum | 5.0 | 250 |
| Small Intestine | 6.0 | 300 |
| Colon | 9.0 | 450 |
| IVP | 4.6 | 230 |
| CT head | 2.0 | 100 |
| CT chest | 8.0 | 400 |
| CT abdomen | 8.0 | 400 |

It is well known in the art that exposure to ionizing radiation, e.g. x-rays, increases a patient's risk of developing certain forms of radiation-induced cancers. Some tissues and organs are especially sensitive to ionizing radiation such as ocular tissues, reproductive organs and breasts. Furthermore, radiation exposure carries an enhanced risk for patients with certain health issues such as patients with pancreatic cancer or patients with an enhanced risk of developing cancer, recent trauma, cirrhosis of the liver, or patients with conditions that require multiple CT scans. Thus, there is a need in the art for methods and devices for conducting low-dose CT, wherein the patient is exposed only to the amount of radiation necessary for the imaging procedure at hand. Previously, some practitioners have achieved lower x-ray exposures by reducing the power of a rotating anode source to a level that is just sufficient for obtaining a suitable image quality. Another approach has been to switch the x-ray source rapidly between two output energies on a millisecond time scale, and using the differing attenuation data to obtain higher resolution images with lower radiation exposure. Others have developed data processing and/or image reconstruction methods that enhance image quality despite lower x-ray beam power, thus enabling practitioners to use lower beam powers.

What is needed is a simple and/or low-cost method for reducing x-ray exposure. Some embodiments may provide such an advantage over the prior art and/or may confer still other benefits over the prior art.

### III. SUMMARY OF THE INVENTION

Some embodiments relate to a low-dose CT apparatus, comprising: a plurality of x-ray sources disposed on a gantry and spaced apart along a z-axis, the sources being configured to produce substantially overlapping fan beams, wherein overlap is substantially complete at a detector surface; at least one x-ray detector configured to receive substantially overlapping fan beams from the plurality of x-ray sources; and a controller in electronic communication with the plurality of x-ray sources, wherein the controller is adapted to switch the plurality of x-ray sources between on and off states such that only one x-ray source is in an on state at any time, and wherein the controller circuit is in electronic communication with the at least one x-ray detector and is adapted to synchronize the at least one x-ray detector with the plurality of x-ray sources such that detected x-rays can be matched to an x-ray source.

According to some embodiments the plurality of x-ray sources comprises one or more of a pair of fixed-anode vacuum tube sources or a pair of rotating anode sources.

According to some embodiments the pair of x-ray sources are operated at a current from 10 to 60 mA.

According to some embodiments the x-ray intensity is from about 2 mSv to about 8 mSv.

According to some embodiments each of the x-ray sources has an on-state of about 10 to 100 ms.

According to some embodiments each of the x-ray sources has an off-state of about 10 to 100 ms.

According to some embodiments only one x-ray source at a time is in an on-state.

According to some embodiments the apparatus operates according to a step and shoot protocol.

Some embodiments further comprise a computing device in electronic data communication with the at least one x-ray detector, wherein the computing device is programmed with an ordered subset expectation optimization image reconstruction protocol.

Other benefits and advantages will become apparent to those skilled in the art to which it pertains upon reading and understanding of the following detailed specification.

### IV. BRIEF DESCRIPTION OF THE DRAWINGS

The invention may take physical form in certain parts and arrangement of parts, embodiments of which will be described in detail in this specification and illustrated in the accompanying drawings which form a part hereof and wherein:

FIG. 1 is a frontal view of an x-ray CT gantry;

FIG. 2A is a top view of a scintillation detector;

FIG. 2B is a side view of the scintillation detector of FIG. 2A;

FIG. 2C is a top view of a pair of adjacent pixels of the scintillation detector shown in FIG. 2A;

FIG. 3 is a frontal view of a pair of x-ray sources with overlapping fan beams; and

FIG. 4 is a drawing of an x-ray tube waveform in relation EKG data.

### V. DETAILED DESCRIPTION OF THE INVENTION

According to some embodiments, a tomographic imaging system comprises a plurality of low-dose x-ray sources. For instance, such source can comprise a pair of x-ray sources that are spaced apart along a z-axis, and are configured to produce substantially overlapping fan beams, wherein overlap is substantially complete at the detector. Furthermore, in some embodiments the anodes are alternately switched between on and off states such that only one anode is on at a time. Although the examples and illustrations herein focus on a pair of anodes, some embodiments can include more than two anodes.

In some embodiments, a suitable x-ray source comprises a fixed-anode vacuum tube. More specifically, some embodiments include a pair of such anodes spaced apart on a gantry in one or more of the θ, φ or z directions. In some embodiments where the sources are spaced apart in the z direction the embodiment can further comprise a collimator disposed on, and in optical communication with, an x-ray scintillation detector, which functions to physically filter out scattered x-ray photons. However, in embodiments where the sources are spaced apart in the θ and/or φ directions software methods, rather than collimators, can be used to correct for and/or filter out scattered x-ray photons.

One of skill in the art will appreciate that operating a fixed anode vacuum tube in continuous mode results in high anode temperatures, which can be detrimental to the lifespan of the tube. Accordingly, some embodiments include a pair of such tubes, wherein one tube is switched on while the other cools in an off-state. Operating the x-ray sources in such manner can provide sufficient x-ray power without unduly shortening the life of the tubes. According to some embodiments, the tubes are oriented to produce overlapping fan beams; therefore, the effect is similar to a single continuous source but with longer tube life.

According to some embodiments a suitable duration for an on-state of an x-ray vacuum tube is between 1 and 200 ms. Other suitable ranges can include one or more of, without limitation, from about 1 to 10 ms, 10 to 20 ms, 20 to 30 ms, 30 to 40 ms, 40 to 50 ms, 50 to 60 ms, 60 to 70 ms, 70 to 80 ms, 80 to 90 ms, 90 to 100 ms, 100 to 110 ms, 110 to 120 ms, 120 to 130 ms, 130 to 140 ms, 140 to 150 ms, 150 to 160 ms, 160 to 170 ms, 170 to 180 ms,180 to 190 ms, 190 to 200 ms, or any combination thereof. Here as elsewhere in the specification and claims, ranges may be combined.

Similarly, according to some embodiments a suitable duration for an off-state of an x-ray vacuum tube is between 1 and 200 ms. Other suitable ranges can include one or more of, without limitation, from about 1 to 10 ms, 10 to 20 ms, 20 to 30 ms, 30 to 40 ms, 40 to 50 ms, 50 to 60 ms, 60 to 70 ms, 70 to 80 ms, 80 to 90 ms, 90 to 100 ms, 100 to 110 ms, 110 to 120 ms, 120 to 130 ms, 130 to 140 ms, 140 to 150 ms, 150 to 160 ms, 160 to 170 ms, 170 to 180 ms,180 to 190 ms, 190 to 200 ms, or any combination thereof.

Furthermore, according to some embodiments, it may be desirable to have only one x-ray source at a time in an on state to eliminate cross talk. Particularly, in some embodiments, the x-ray detector cannot distinguish between the individual sources. Accordingly, the detector must be synchronized with the respective sources in order to distinguish between them.

Suitable x-ray tube currents can be from about 1 to 60 mA. More specifically, suitable ranges can include one or more of, without limitation, 1 to 5 mA, 5 to 10 mA, 10 to 15 mA, 15 to 20 mA, 20 to 25 mA, 25 to 30 mA, 30 to 35 mA, 35 to 40 mA, 40 to 45 mA, 45 to 50 mA, 50 to 55 mA, 55 to 60 mA or any combination thereof

In some embodiments the waveform describing the operation of an x-ray tube can comprise a simple square waveform with a constant period and amplitude. However, in some embodiments the waveform can be a composite of a plurality of waveforms. For example, in an EKG gated embodiment a constant-period square waveform can be synchronized with diastole, while the onset of systole can cause both tubes to be switched to an off state until the heart returns to diastole. According to such embodiments, the EKG can be used to trigger the tubes to enter an off state. For instance, when the system detects the P wave indicating the onset of systole, the tubes may be turned off. Thus, the system only collects data during diastole, which enables higher quality image reconstruction.

Similarly, some embodiments can include triggering an off state by sensing other physiological parameters. For instance, it may be desirable to stop collecting data during certain phases of the breathing cycle, or during coughing spasms. Thus, the rise and fall of the chest can be detected, for instance through laser ranging methods such as Light Detection and Ranging (i.e. LIDAR) or other known methods. Optionally, some embodiments can include a traversing titled gantry, a LIDAR chest/body monitor, and/or prospective adaptive x-ray exposure control. Some embodiments can also include an ordered subset expectation maximization (OSEM) image reconstruction algorithm in place of a filtered back projection algorithm. Particularly, in some embodiments improved image quality can be achieved using an OSEM method.

When operated according to some embodiments of the invention, an x-ray CT scan can reduce the exposure of a patient to ionizing radiation by up to 50% or about 1 to 2 mSv.

In some embodiments an x-ray vacuum tube source comprises a focal spot size from about 1 μm² to about 2000 μm².

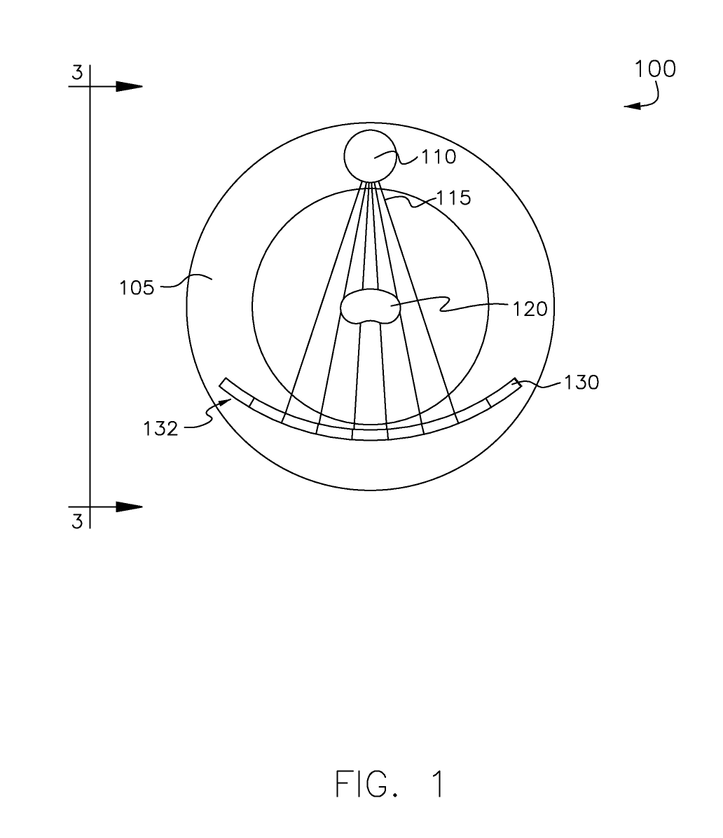

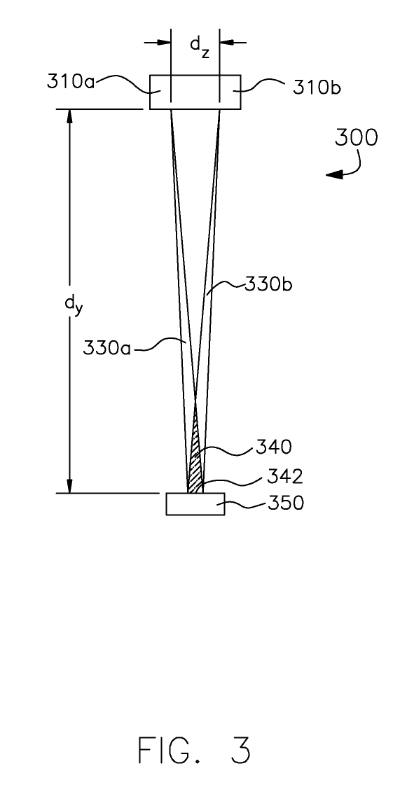

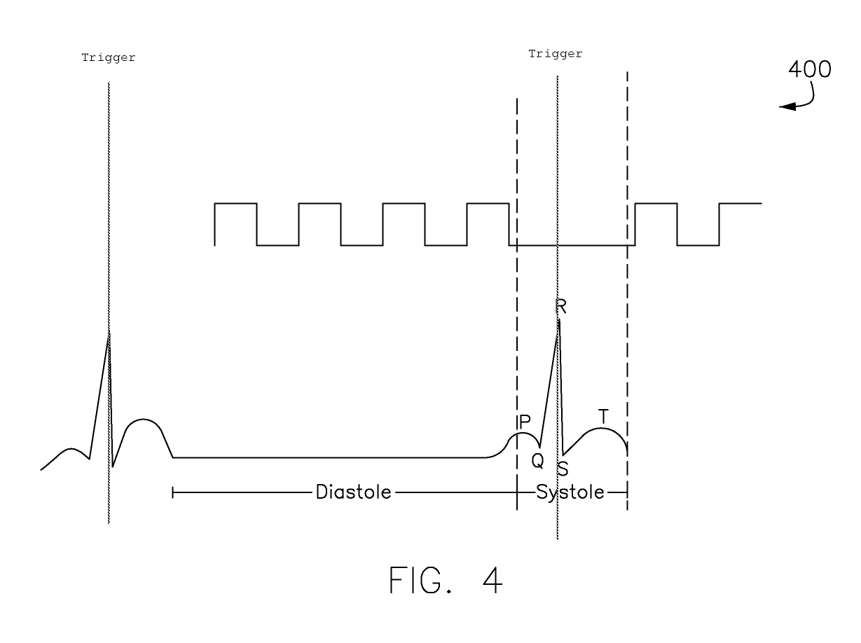

Turning to FIG. 1, a front view of an embodiment 100 is shown comprising a CT gantry 105 with a dual x-ray source 110 mounted in an upper position. According to this view, the x-ray source 110 emanates x-ray fan beams 115. The fan beams 115 are spaced apart along the z-axis, i.e. in the plane of the page, and therefore are not distinguishable from one another in this view. With continuing reference to FIG. 1, a cross sectional view of a patient 120 is shown attenuating the beams 115, which are then detected by a plurality of x-ray scintillation detectors 130 arranged in an arc 132. In some embodiments, the detectors 130 can comprise, without limitation, a 64×64 pixelated array of LYSO crystals as shown in FIG. 3.

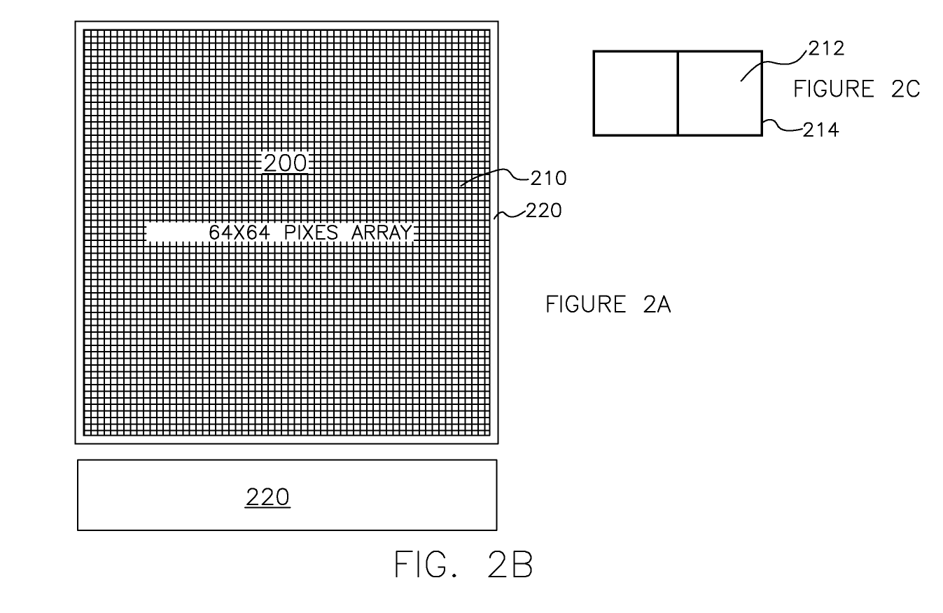

FIG. 2A-C shows an x-ray scintillation detector 200 consistent with at least some embodiments of the invention. The detector 200 comprises a 64×64 array of LYSO scintillation crystals 210 bounded by a perimeter comprising a brass housing 220. According to FIG. 2C individual LYSO crystals 212 are separated from one another by UV reflective septa 214.

FIG. 3 shows an embodiment 300 comprising a pair of fixed anode x-ray tubes 310a, 310b. The anodes of tubes 310a and 310b are spaced apart by a distance d_{z. The tubes 310}a, 31b produce fan beams 330a, 330b, which form an overlapping region 340 and impinge on a common overlapping area 342 of a detector 350. According to some embodiments, the fan beams can impinge upon the entire surface of the scintillation detector(s); however, in some embodiments the fan beams can impinge upon less than the entire surface, or may even extend somewhat beyond the detector(s) surface.

The embodiments have been described, hereinabove and shown in the various drawing views, which are included for purposes of illustrating embodiments of the invention and not for limiting the same. Thus, it will be apparent to those skilled in the art that the above methods and apparatuses may incorporate changes and modifications without departing from the general scope of this invention. Accordingly, it is intended to include all such modifications and alterations insofar as they come within the scope of the appended claims or the equivalents thereof

Having thus described the invention, it is now claimed:

I/we claim:

1. A low-dose CT apparatus, comprising:
a plurality of x-ray sources disposed on a gantry and spaced apart along a z-axis, the sources being configured to produce substantially overlapping fan beams, wherein overlap is substantially complete at a detector surface;
at least one x-ray detector configured to receive substantially overlapping fan beams from the plurality of x-ray sources; and
a controller in electronic communication with the plurality of x-ray sources, wherein the controller is adapted to switch the plurality of x-ray sources between on and off states such that only one x-ray source is in an on state at any time, and wherein the controller circuit is in electronic communication with the at least one x-ray detector and is adapted to synchronize the at least one x-ray detector with the plurality of x-ray sources such that detected x-rays can be matched to an x-ray source.

2. The apparatus of claim 1, wherein the plurality of x-ray sources comprises one or more of a pair of fixed-anode vacuum tube sources or a pair of rotating anode sources.

3. The apparatus of claim 2, wherein the pair of x-ray sources is operated at a current from 10 to 60 mA.

4. The apparatus of claim 2, wherein the x-ray intensity is from about 2 mSv to about 8 mSv.

5. The apparatus of claim 2, wherein each of the x-ray sources has an on-state of about 10 to 100 ms.

6. The apparatus of claim 5, wherein each of the x-ray sources has an off-state of about 10 to 100 ms.

7. The apparatus of claim 6, wherein only one x-ray source at a time is in an on-state.

8. The apparatus of claim 1, wherein the apparatus operates according to a step and shoot protocol or a spiral scan protocol.

9. The apparatus of claim 1, further comprising a computing device in electronic data communication with the at least one x-ray detector, wherein the computing device is programmed with an ordered subset expectation optimization image reconstruction protocol.

10. A low-dose CT apparatus, comprising:
a pair of fixed-anode vacuum tube sources disposed on a gantry and spaced apart along a z-axis, the sources being configured to produce substantially overlapping fan beams, wherein overlap is substantially complete at a detector surface, and, wherein each of the x-ray sources defines a 50% duty cycle having an on-state of about 10 to 100 ms;
at least one x-ray detector configured to receive substantially overlapping fan beams from the plurality of x-ray sources; and
a controller in electronic communication with the plurality of x-ray sources, wherein the controller is adapted to switch the plurality of x-ray sources between on and off states such that only one x-ray source is in an on state at any time, and wherein the controller circuit is in electronic communication with the at least one x-ray detector and is adapted to synchronize the at least one x-ray detector with the plurality of x-ray sources such that detected x-rays can be matched to an x-ray source.

11. The apparatus of claim 10, wherein only one x-ray source at a time is in an on-state.

12. The apparatus of claim 10, wherein the apparatus operates according to a step and shoot protocol or a spiral scan protocol.

13. The apparatus of claim 10, further comprising a computing device in electronic data communication with the at least one x-ray detector, wherein the computing device is programmed with an ordered subset expectation optimization image reconstruction protocol.
